(19) United States
(12) Patent Application Publication
(10) Pub. No.: US 2015/0352078 A1
(43) Pub. Date: Dec. 10, 2015

(54) PHARMACEUTICAL COMPOSITION FOR DISEASES CAUSED BY PATHOGENIC MICROORGANISMS SUCH AS CANDIDA

(71) Applicants: POLA PHARMA INC., Tokyo (JP); NIHON NOHYAKU CO., LTD., Tokyo (JP)

(72) Inventors: Tsuyoshi Shimamura, Yokohama-shi (JP); Yoshiyuki Miyata, Tokyo (JP); Makoto Gotoh, Tokyo (JP)

(21) Appl. No.: 14/760,887

(22) PCT Filed: Dec. 27, 2013
(86) PCT No.: PCT/JP2013/085344

§ 371 Date: Jul. 14, 2015

(30) Foreign Application Priority Data
Jan. 28, 2013 (JP) 2013-013506
Apr. 2, 2013 (JP) 2013-076596

(51) Int. Cl. A61K 31/4178

(52) U.S. Cl. CPC A61K 31/4178

(57) ABSTRACT

An object is to provide means for precisely treating vaginitis caused by intracellular parasite, protozoa, and/or fungus. A pharmaceutical composition for vaginitis, comprising a compound represented by the following general formula (1) as an active ingredient: (1) (In the formula, R represents a halogen atom or a hydrogen atom, and X represents a halogen atom.)

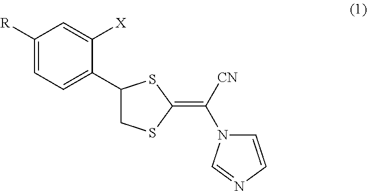

### TECHNICAL FIELD

The present invention relates to a pharmaceutical composition. In particular, the present invention relates to a pharmaceutical composition preferably usable for vaginitis or colpitis.

### BACKGROUND ART

Patients infected with vaginitis (colpitis) are increased in relation to STD in recent years. As for Chlamydia vaginitis, for example, it is also reported that about 70% of specimens had the pathogen according to a fixed point investigation performed by a certain public health center in the Tokyo metropolitan area (see, for example, http://idsc.tokyo-eiken.go.jp/diseases/sti/). Trichomonas vaginitis as well as Chlamydia vaginitis is also classified into STD. It is said that Trichomonas vaginitis is increased as accompanied with the increase in illicit sexual act in the same manner as Chlamydia vaginitis. Tetracycline, new quinolone, and macrolide antibiotic are effective on Chlamydia vaginitis. On the other hand, the therapeutic agent is only metronidazole for Trichomonas vaginitis. Further, as for the oral administration of metronidazole, the response rate is low with respect to the vaginitis, it is difficult to exterminate protozoa especially in vagina, and it is inevitable to rely on any vaginal tablet. However, it is said that the efficacy of the vaginal tablet is low to exterminate protozoa from outer labia. In this sense, any therapeutic means, which is effective on Trichomonas vaginitis, has not been obtained in the present circumstances, although such means is demanded. The situation as described above also arises identically in relation to Chlamydia provided that the therapeutic agent is merely changed to tetracycline antibiotic, new quinolone antibiotic, and/or macrolide antibiotic. It is affirmed that any technique, which can cure or treat vaginitis caused by the pathogenic microorganism by means of one medicament (agent or drug), has not been obtained in the present circumstances, although such a technique is demanded.

Further, in recent years, it has been confirmed that fungi such as Candida and Aspergillus co-exist in many cases in relation to Trichomonas vaginitis (see, for example, Non-Patent Document 1 and Non-Patent Document 2). Even when Trichomonas vaginitis is cured, it is not rare that Candida vaginitis or Aspergillus vaginitis is newly caused. That is, any agent or drug, which can also treat or cure fungal vaginitis simultaneously with Trichomonas vaginitis, is not obtained, although the agent or drug is demanded. In the experience of the present inventors, the co-existence of Candida albicans was confirmed for 71 strains of clinically isolated 143 strains of Trichomonas vaginalis. In other words, about 50% of Trichomonas strains co-existed with Candida, in other words, caused the multiple infection in vaginitis. The situation as described above also arises in relation to Chlamydia in the same manner as described above. The multiple infection of Chlamydia and Trichomonas and the multiple infection of Chlamydia and fungus are present. Such multiple infection is considered to be one of the factors to which the most careful attention should be paid when the medical treatment is performed.

On the other hand, it is known that a compound such as lanoconazole or luliconazole, which is represented by the general formula (1), has the antifungal action (see, for example, Patent Document 1, Patent Document 2, and Patent Document 3). It has been known that lanoconazole and lysozyme or clotrimazole are combined and used for the medical treatment of Candida vaginitis (see, for example, Patent Document 4). However, nothing has been known at all about the fact that the compound as described above is singly used as an active ingredient and the compound as described above is used to cure or prevent vaginitis caused by any fungus such as Candida, Aspergillus or the like, vaginitis caused by protozoa such as Trichomonas or the like, vaginitis caused by intracellular parasite such as Chlamydia or the like, and a combination of vaginitis caused by any fungus, protozoa such as Trichomonas or the like, and intracellular parasite such as Chlamydia or the like.

Any compound is scarcely known, which is known as an antifungal agent and which simultaneously has an antiprotozoal action and an anticlamydial action. For example, it is known that bifonazole, miconazole, amorolfin, and butenafine, which are known as antifungal agents against athlete's foot or the like, do not have the antiprotozoal action and the anticlamydial action.

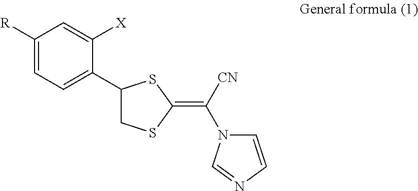

(In the formula, R represents a hydrogen atom or a halogen atom, and X represents a halogen atom.)

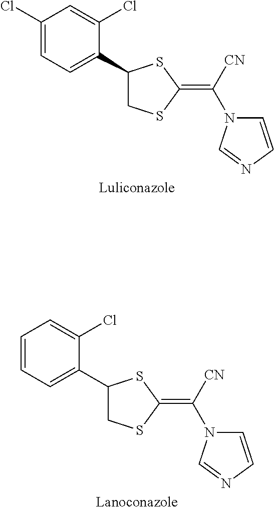 [SMILES: N#C/C(=C1/SC[C@@H](c2ccc(Cl)cc2Cl)S1)n1ccnc1 ; N#C/C(=C1/SCC(c2ccccc2Cl)S1)n1ccnc1]

### PRECEDING TECHNICAL DOCUMENTS

### Patent Documents

Patent Document 1: JP2007-84496A;
Patent Document 2: JP2009-515958W;
Patent Document 3: JP09-100279A;
Patent Document 4: JP08-198773A.

### Non-Patent Documents

Non-Patent Document 1: Zdrodowska-Stefanow B, Klosowska W M, Ostaszewska-Puchalska I, Bulhak-Koziol V, Kotowicz B; “Ureaplasma urealyticum and Mycoplasma hominis infection in women with urogenital diseases.” Adv Med Sci. 2006; 51:250-3.
Non-Patent Document 2: Mittal A, Rastogi S, Reddy B S, Verma S, Salhan S, Gupta E; “Enhanced immunocompetent cells in chlamydial cervicitis.” J Reprod Med. 2004; 49(8):671-7

### SUMMARY OF THE INVENTION

### Technical Problem

The present invention has been made under the circumstances as described above, an object of which is to provide means for precisely treating vaginitis caused by intracellular parasite, protozoa, and/or fungus.

### Solution to Problem

Taking the foregoing circumstances into consideration, the present inventors have repeatedly performed diligent researches and efforts in order to seek for means for precisely treating vaginitis caused by intracellular parasite, protozoa, and/or fungus. As a result, it has been found out that the compound such as luliconazole and lanoconazole, which is represented by the general formula (1) described above, has the action or function to inhibit the growth of intracellular parasite such as Chlamydia or the like, protozoa such as Trichomonas or the like, and fungus such as Candida, Aspergillus or the like. It has been found out that vaginitis, which is caused by intracellular parasite, protozoa, and/or fungus, can be precisely treated by using the compound as described above as an active ingredient. Thus, the invention has been completed. That is, the present invention is as follows.

<1> A pharmaceutical composition for vaginitis, comprising a compound represented by the following general formula (1) as an active ingredient:

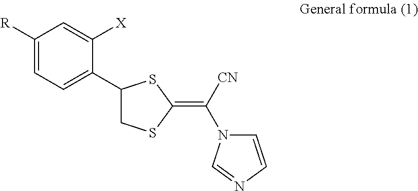

(In the formula, R represents a halogen atom or a hydrogen atom, and X represents a halogen atom.)

<2> The pharmaceutical composition for vaginitis as defined in <1>, wherein the compound represented by the general formula (1) is luliconazole or lanoconazole:

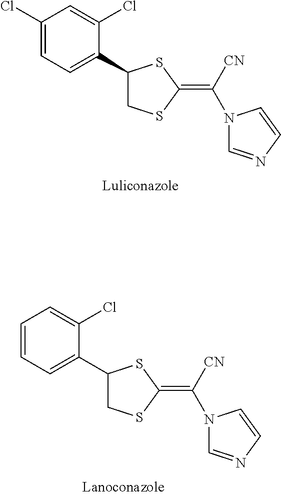

<3> The pharmaceutical composition for vaginitis as defined in <1> or <2>, wherein the vaginitis is caused by a pathogen selected from intracellular parasite, protozoa, and fungus.

<4> The pharmaceutical composition for vaginitis as defined in <3>, wherein the protozoa is protozoa belonging to genus Trichomonas.

<5> The pharmaceutical composition for vaginitis as defined in <3> or <4>, wherein the fungus is fungus belonging to genus Candida and/or fungus belonging to genus Aspergillus.

<6> The pharmaceutical composition for vaginitis as defined in any one of <3> to <5>, wherein the intracellular parasite is intracellular parasite belonging to genus Chlamydia.

<7> The pharmaceutical composition for vaginitis as defined in any one of <1> to <6>, wherein a content of the compound represented by the general formula (1) is 1 to 60% by mass with respect to a total amount of the pharmaceutical composition.

<8> The pharmaceutical composition for vaginitis as defined in any one of <1> to <7>, further containing 40 to 99% by mass of an arbitrary component for preparing a pharmaceutical preparation.

<9> The pharmaceutical composition for vaginitis as defined in any one of <1> to <8>, wherein the pharmaceutical composition is a suppository, a tablet, or a gel.

<10> An antiprotozoal agent, comprising a compound represented by a general formula (1) as an active ingredient.

<11> The antiprotozoal agent as defined in <10>, wherein the protozoa is protozoa belonging to genus Trichomonas.

<12> An anti-intracellular parasite agent, comprising a compound represented by a general formula (1) as an active ingredient.

<13> The anti-intracellular parasite agent as defined in <12>, wherein the intracellular parasite is intracellular parasite belonging to genus Chlamydia.

### Advantageous Effects of Invention

According to the present invention, it is possible to provide means for precisely treating vaginitis caused by intracellular parasite, protozoa, and/or fungus.

### BRIEF DESCRIPTION OF THE DRAWINGS

FIG. 1 shows drawings (photographs) illustrating observation results of Chlamydia inclusion bodies after the luliconazole treatment, as obtained by the fluorescence staining by using Chlamydia FA reagent “Seiken”. Panel (A) shows the observation result of Chlamydia inclusion bodies after a treatment with 8 μg/mL of luliconazole, panel (B) shows the observation result of Chlamydia inclusion bodies after a treatment with 16 μg/mL of luliconazole, and panel (C) shows the observation result of Chlamydia inclusion bodies after a treatment with 32 μg/mL of luliconazole. In (A) and (B), dot-shaped Chlamydia inclusion bodies stained apple green were observed. No inclusion body was found in (C).

### DESCRIPTION OF EMBODIMENTS

### <1> Compound Represented by General Formula (1)

The pharmaceutical composition of the present invention is characterized in that the pharmaceutical composition contains the compound represented by the general formula (1) and the pharmaceutical composition is usable for vaginitis. In the general formula (1)′, the group represented by R is a hydrogen atom or a halogen atom. The halogen atom can be preferably exemplified, for example, by chlorine atom, bromine atom, fluorine atom, and iodine atom. The group represented by R is especially preferably a hydrogen atom or a chlorine atom. The group represented by X represents a halogen atom. The halogen atom can be preferably exemplified, for example, by chlorine atom, bromine atom, fluorine atom, and iodine atom. The group represented by X is especially preferably a chlorine atom. The compound represented by the general formula (1) is especially preferably luliconazole (R═X═Cl; (R)-(−)-(E)-[4-(2,4-dichlorophenyl)-1,3-dithiolane-2-ylidene]-1-imidazolyl acetonitrile) and lanoconazole (R═H, X═Cl; 4-(2-chlorophenyl)-1,3-dithiolane-2-ylidene-1-imidazolyl acetonitrile), and luliconazole is especially preferred. The compound as described above suppresses the growth of intracellular parasite such as Chlamydia or the like and protozoa such as Trichomonas or the like, and the compound as described above also suppresses the growth of fungus such as Candida, Aspergillus or the like.

The compound as described above can be synthesized, for example, in accordance with a method described in JP60-218387A. That is, 1-cyanomethylimidazole and carbon disulfide are reacted to obtain a compound of (III) which is reacted with a compound of the general formula (II) having a leaving group. Thus, it is possible to obtain the compound represented by the general formula (1) as described above. The leaving group as described above can be preferably exemplified, for example, by methanesulfonyloxy group, benzenesulfonyloxy group, p-toluenesulfonyloxy group, and halogen atom.

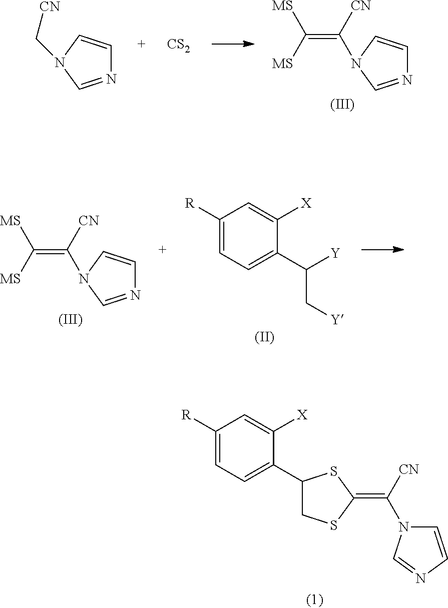

In the formula, Y, Y′ represent the leaving group such as methanesulfonyloxy group, benzenesulfonyloxy group, p-toluenesulfonyloxy group, and halogen atom, and M represents alkali metal.

In order that the compound represented by the general formula (1) exhibits the anti-intracellular parasite action, the anti-protozoa action (antiprotozoal action), and the antifungal action, it is preferable that the compound represented by the general formula (1) is contained in the pharmaceutical composition of the present invention usually by 0.5 to 80% by mass and more preferably by 1 to 60% by mass with respect to the total amount of the pharmaceutical composition.

### <2> Pharmaceutical Composition of the Present Invention

The pharmaceutical composition of the present invention can contain any arbitrary component for preparing a pharmaceutical preparation, other than the compound represented by the general formula (1) described above. It is preferable that the component for preparing the pharmaceutical preparation is the residual part or the balance of the compound represented by the general formula (1). The component for preparing the pharmaceutical preparation is usually 20 to 99.5% by mass and preferably 40 to 99% by mass in a total amount with respect to the total amount of the pharmaceutical composition of the present invention.

The component for preparing the pharmaceutical preparation can be preferably exemplified as follows, for example, in the case of the tablet. That is, it is possible to preferably exemplify excipients such as lactose, croscarmellose and the like; alkali agents such as sodium carbonate, sodium hydrogencarbonate and the like; acid agents such as citric acid, lactic acid, tartaric acid and the like; coating agents such as hydroxypropyl methylcellulose, triethyl citrate and the like; binding agents such as gum arabic and the like; disintegrating agents such as starch, hydroxypropyl cellulose, croscarmellose and the like; sugar coating agents such as sucrose, maltitol and the like; surfactants such as POE-cured castor oil, POE sorbitan fatty acid ester and the like; plasticizers such as triethyl citrate, caprylic capric monoglyceride, diethylene glycol monoethyl ether and the like; and lubricants such as magnesium stearate, talc and the like. As for the tablet, it is also possible to adopt a form of vaginal tablet as well as tablet for oral administration. When the form of vaginal tablet is adopted, it is preferable to provide a foam tablet or effervescent tablet obtained by the tablet making after coating alkaline granules and acidic granules in order to facilitate the disintegration performance.

It is also possible to preferably exemplify, for example, the suppository or the gel to be directly administered into the vagina. In the case of the suppository, it is possible to preferably exemplify hydrocarbons such as Vaseline, solid paraffin, microcrystalline wax, liquid paraffin and the like; esters such as Witepsol, spermaceti, carnauba wax, Japan tallow, beeswax, jojoba oil, octyldodecyl oleate and the like; triglycerides such as olive oil, coconut oil, glyceryl triisostearate, glyceryl tristearate and the like; higher alcohols such as oleyl alcohol, behenyl alcohol, stearyl alcohol and the like; lipophilic surfactants such as monoglyceryl stearate, monoglyceryl oleate, sorbitan fatty acid ester and the like; and solvents such as N-alkyl-2-pyrrolidone, alkylene carbonate, benzyl alcohol and the like. As for the gel, it is possible to preferably exemplify polyhydric alcohols such as glycerol, 1,3-butanediol, propylene glycol, polyethylene glycol and the like; volatile solvents such as ethanol, acetone, methyl ethyl ketone and the like; and thickening/gelling agents such as (ammonium acryloyldimethyltaurate/VP) copolymer, (ammonium acryloyldimethyltaurate/beheneth-25 methacrylate) crosspolymer, ethyl cellulose, carboxyvinyl polymer, hydroxypropyl methylcellulose and the like.

The pharmaceutical composition of the present invention can be produced on the basis of any conventional method by using the compound represented by the general formula (1) described above and arbitrary components for preparing the pharmaceutical preparation.

As for the pharmaceutical composition of the present invention, it is possible to use any one of the pharmaceutical preparations as described above. It is especially preferable to use the pharmaceutical preparation having the form capable of being directly administered into the vagina. The compound of the present invention is not absorbed into the body in the vagina, and the effect thereof is not decreased, unlike metronidazole. Therefore, the compound of the present invention can sufficiently exhibit the effect in relation to the mode as described above.

A preferred mode of application can be appropriately selected while considering, for example, the body weight, the age, the sexuality, and the symptoms or condition of the patient. However, in the ordinary case of an adult, it is appropriate to perform the administration once or several times per day in the vagina so that the administration amount of the compound represented by the general formula (1) is 0.1 to 10 g, and such a treatment is performed for about 1 day to 3 weeks.

The compound represented by the general formula (1) has the anti-intracellular parasite action, the anti-protozoa action (antiprotozoal action), and the antifungal action against the intracellular parasite, the protozoa, and the fungus. The pharmaceutical composition of the present invention has been achieved on the basis of such knowledge acquired by the present inventors.

That is, the disease, to which the pharmaceutical composition of the present invention is applicable, can be the vaginitis which is caused by a pathogen selected from intracellular parasite, protozoa, and/or fungus (for example, vaginitis diagnosed that the pathogen is intracellular parasite, protozoa, and/or fungus).

In this context, the “pharmaceutical composition for vaginitis caused by the pathogen of protozoa of the present invention” can be applied to the vaginitis in which the pathogen is protozoa and the vaginitis in which the pathogen is protozoa and intracellular parasite and/or fungus. In consideration of the present circumstances in which, for example, there are many cases of the co-existence of protozoa and intracellular parasite and/or fungus or the secondary infection of protozoa, it is also preferable to apply the “pharmaceutical composition for vaginitis caused by the pathogen of protozoa of the present invention” to the vaginitis caused by the pathogen of fungus and/or intracellular parasite, in view of the suppression of any potential infection of protozoa and the prevention of any secondary infection. Further, the application to the vaginitis caused by the pathogen of fungus and/or intracellular parasite for the purpose as described above is also included in the scope of the present invention.

Further, the “pharmaceutical composition for vaginitis caused by the pathogen of fungus of the present invention” can be applied to the vaginitis in which the pathogen is fungus and the vaginitis in which the pathogen is fungus and protozoa and/or intracellular parasite. In consideration of the present circumstances in which, for example, there are many cases of the co-existence of fungus and protozoa and/or intracellular parasite or the secondary infection of fungus, it is also preferable to apply the “pharmaceutical composition for vaginitis caused by the pathogen of fungus of the present invention” to the vaginitis caused by the pathogen of protozoa and/or intracellular parasite, in view of the suppression of any potential infection of fungus and the prevention of any secondary infection. Further, the application to the vaginitis caused by the pathogen of protozoa and/or intracellular parasite for the purpose as described above is also included in the scope of the present invention.

Similarly, it is also possible to treat intracellular parasite. That is, the “pharmaceutical composition for vaginitis caused by the pathogen of intracellular parasite of the present invention” can be applied to the vaginitis in which the pathogen is intracellular parasite and the vaginitis in which the pathogen is intracellular parasite and fungus and/or protozoa. In consideration of the present circumstances in which, for example, there are many cases of the co-existence of intracellular parasite and fungus and/or protozoa or the secondary infection of intracellular parasite, it is also preferable to apply the “pharmaceutical composition for vaginitis caused by the pathogen of intracellular parasite of the present invention” to the vaginitis caused by the pathogen of fungus and/or protozoa, in view of the suppression of any potential infection of intracellular parasite and the prevention of any secondary infection. Further, the application to the vaginitis caused by the pathogen of fungus and/or protozoa for the purpose as described above is also included in the scope of the present invention.

The “pharmaceutical composition for vaginitis caused by the pathogen of intracellular parasite, protozoa, and fungus of the present invention” can be applied not only to the vaginitis in which the pathogen is intracellular parasite, protozoa, and fungus but also to the vaginitis in which the pathogen is protozoa, the vaginitis in which the pathogen is fungus, and the vaginitis in which the pathogen is intracellular parasite, in view of the suppression of any potential infection of intracellular parasite, protozoa, or fungus and the prevention of any secondary infection. Further, the application to the vaginitis caused by the pathogen of protozoa, the vaginitis caused by the pathogen of fungus, and the vaginitis caused by the pathogen of intracellular parasite for the purpose as described above is also included in the scope of the present invention.

The fungus, which is the objective or target of the present invention, is not specifically limited, which is exemplified, for example, by fungi belonging to the genus Candida such as Candida albicans and the like and the genus Aspergillus.

The protozoa, which is the objective or target of the present invention, is not specifically limited, which is exemplified, for example, by protozoas belonging to the genus Trichomonas such as Trichomonas vaginalis and the like.

The intracellular parasite, which is the objective or target of the present invention, is not specifically limited, which is exemplified, for example, by intracellular parasites belonging to the genus Chlamydia such as Chlamydia trachomatis and the like.

### EXAMPLES

The present invention will be explained in further detail below as exemplified by Examples. However, the present invention is not limited to Examples described below.

### Example 1

The effect on Trichomonas vaginalis was investigated for luliconazole of the compound represented by the general formula (1). That is, 5×10⁶ cells of clinically isolated Trichomonas vaginalis were seeded in Trichomonas medium F (6.5 mL, contained in tube) produced by Fujiyakuhin Co., Ltd. containing Neutral Red as a marker, and the preculture was carried out for 72 hours (preculture). It was confirmed that Trichomonas grew, the acid was actively produced, and Neutral Red was changed to be yellow. After that, the preculture was added to Trichomonas medium F by every 100 μL in order to carry out the main culture, to which 0.5 mL of a test solution was added. In this situation, the number of protozoas in the solution of preculture was 1.5×10⁵ cells/mL. Three series of test solutions were prepared, in which the luliconazole concentrations were 200 μM (final concentration: 35.2 μM), 100 μM (final concentration: 17.6 μM), and 50 μM (final concentration: 8.8 μM), as luliconazole dissolved in 10% methanol saline solution. 0.5 mL of vehicle was added as a control. As for the vehicle, 10% methanol saline solution (final concentration: 0 μM) was used. Stirring was sufficiently performed after the addition, followed by culturing at 37° C. for 72 hours. After the completion of the cultivation, the color was discriminated, and the state of protozoas was observed by using an inverted microscope. Results are shown in Table 1. Accordingly, it is appreciated that luliconazole inhibits the growth of Trichomonas at 8.8 μM. In other words, it has been revealed that luliconazole is a substance which can inhibit the growth with respect to Trichomonas and which is clinically applicable, except for metronidazole. Further, it is also revealed that the minimum growth inhibitory concentration (MIC) is in the vicinity of 8.8 μM.

TABLE 1

| Final | | |
|---|---|---|
| concentration | Color | Result of microscopic observation |
| 35.2 μM | red | no protozoa was observed |
| 17.6 μM | red | no protozoa was observed |
| 8.8 μM | yellow | protozoas were observed slightly |
| 0 μM | yellow | large number of protozoas were |
| | | observed |

### Example 2

The same or equivalent investigation as that of Example 1 was performed while changing luliconazole to lanoconazole. As a result, it becomes clear that lanoconazole also inhibits the growth of Trichomonas as well as luliconazole. It has been revealed that lanoconazole is a substance which can inhibit the growth with respect to Trichomonas and which is clinically applicable, except for metronidazole. Further, it is revealed that the minimum growth inhibitory concentration (MIC) is in the vicinity of 17.6 μM.

TABLE 2

| Final | | |
|---|---|---|
| concentration | Color | Result of microscopic observation |
| 35.2 μM | red | no protozoa was observed |
| 17.6 μM | red | protozoas were observed slightly |
| 8.8 μM | yellow | large number of protozoas were |
| | | observed |
| 0 μM | yellow | large number of protozoas were |
| | | observed |

### Example 3

### In Vitro Antifungal Activity on Candida albicans

The minimum growth inhibitory concentration (MIC) was measured by means of the broth microdilution method (medicament ×2 dilution series) based on the use of BPMI 1640 medium (pH 7.0) buffered with 0.165 M morpholinopropanesulfonic acid. 100 μL of test microorganism yeast cells/sterilized physiological saline suspensions (1 to 5×10³ cells/mL) and 100 μL of media previously added with respective compounds and medium not added with, compounds as a control were dispensed into respective wells of flat-bottom microculture plate. After performing the cultivation at 35° C. for 48 hours, the culture turbidities of the respective wells were measured at 630 nm to determine the minimum growth inhibitory concentration (MIC₈₀: μg/mL) as the minimum concentration of the compound at which the growth inhibition of 80% was exhibited with respect to the growth of the microorganism in the control culture (measured as the suspension). Results are shown in Table 3. It is appreciated that the excellent antifungal activity is exhibited in any case. Considering this fact in combination with Examples 1 and 2, it is clear that it is possible to simultaneously inhibit the growth of protozoa such as Trichomonas and the growth of fungus such as Candida by using the compound represented by the general formula (1).

TABLE 3

| Microbial strain | Luliconazole | Lanoconazole |
|---|---|---|
| IFO0197 | 0.0625 | 0.125 |
| IFO0579 | 0.0313 | 0.0625 |
| IFO1269 | 0.0625 | 0.0625 |
| TIMM3164 | 0.5 | 0.5 |

### Example 4

Vaginal tablets were manufactured in accordance with the following formulation. That is, Part A and Part B were granulated into granules respectively, and coating was performed while spraying hydroxypropyl methylcellulose and triethyl citrate dissolved in ethanol. After the completion of the coating, the blowing was performed with warm air at 40° C. to perform the drying. After the drying, granules A and granules B were mixed, followed by being manufactured into effervescent tablets in accordance with the tablet making process.

TABLE 4

| | |
|---|---|
| A | |
| (Granule component) | |
| Citric acid | 15 parts by mass |
| Lactic acid | 10 parts by mass |
| Luliconazole | 20 parts by mass |
| Hydroxypropyl cellulose | 0.5 part by mass |
| (Coating agent) | |
| Hydroxypropyl methylcellulose | 4 parts by mass |
| Triethyl citrate | 0.5 part by mass |
| B | |
| (Granule component) | |
| Sodium hydrogencarbonate | 25 parts by mass |
| Croscarmellose | 20 parts by mass |
| Hydroxypropyl cellulose | 0.5 part by mass |
| (Coating agent) | |
| Hydroxypropyl methylcellulose | 4 parts by mass |
| Triethyl citrate | 0.5 part by mass |

### Example 5

Vaginal tablets were manufactured in accordance with the following formulation. That is, Part A and Part B were granulated into granules respectively, and coating was performed while spraying hydroxypropyl methylcellulose and triethyl citrate dissolved in ethanol. After the completion of the coating, the blowing was performed with warm air at 40° C. to perform the drying. After the drying, granules A and granules B were mixed, followed by being manufactured into effervescent tablets in accordance with the tablet making process.

TABLE 5

| | |
|---|---|
| A | |
| (Granule component) | |
| Citric acid | 15 parts by mass |
| Lactic acid | 10 parts by mass |
| Lanoconazole | 20 parts by mass |
| Hydroxypropyl cellulose | 0.5 part by mass |
| (Coating agent) | |
| Hydroxypropyl methylcellulose | 4 parts by mass |
| Triethyl citrate | 0.5 part by mass |
| B | |
| (Granule component) | |
| Sodium hydrogencarbonate | 25 parts by mass |
| Croscarmellose | 20 parts by mass |
| Hydroxypropyl cellulose | 0.5 part by mass |
| (Coating agent) | |
| Hydroxypropyl methylcellulose | 4 parts by mass |
| Triethyl citrate | 0.5 part by mass |

### Example 6

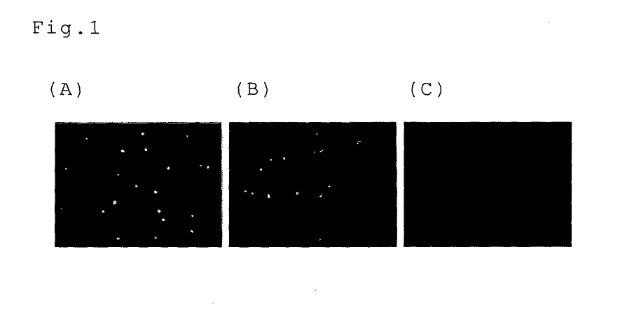

The anti-intracellular parasite action was investigated by using Chlamydia trachomatis (D/UW3/Cx). That is, Chlamydia trachomatis was cultured in the presence of ×2 dilution series of 8 to 64 μg/ml of luliconazole by using HeLa 229 cells as the host. MEM added with 8% thermally inactivated FBS, to which 1 μg/ml of cyclohexamide was added, was used as the medium, and the culture was performed for 72 hours in 5% carbon dioxide gas at 37° C. After the culture, Chlamydia inclusion bodies were subjected to the fluorescent staining to be apple green with Chlamydia FA reagent “Seiken” (produced by DENKA SEIKEN Co., Ltd.), and the observation was performed by using a fluorescence microscope. Results are shown in FIG. 1. Accordingly, it is appreciated that MIC of luliconazole with respect to Chlamydia trachomatis is 32 μg/ml.

### INDUSTRIAL APPLICABILITY

The present invention is applicable to pharmaceuticals.

1. A pharmaceutical composition for vaginitis, comprising a compound represented by the following general formula (1) as an active ingredient: 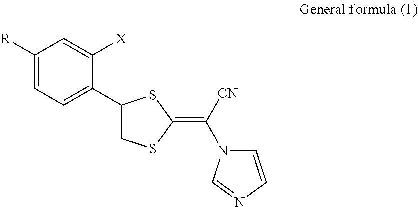
wherein, R represents a halogen atom or a hydrogen atom, and X represents a halogen atom.

2. The pharmaceutical composition for vaginitis according to claim 1, wherein the compound represented by the general formula (1) is luliconazole or lanoconazole:

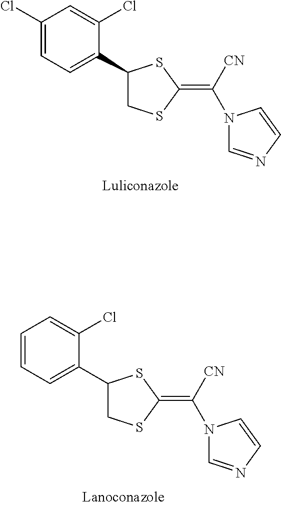

3. The pharmaceutical composition for vaginitis according to claim 1, wherein the vaginitis is caused by a pathogen selected from the group consisting of intracellular parasite, protozoa, and fungus.

4. The pharmaceutical composition for vaginitis according to claim 3, wherein the pathogen is protozoa and wherein the protozoa are protozoa belonging to genus Trichomonas.

5. The pharmaceutical composition for vaginitis according to claim 3, wherein the pathogen is fungus and wherein the fungus is fungus belonging to genus Candida and/or fungus belonging to genus Aspergillus.

6. The pharmaceutical composition for vaginitis according to claim 3, wherein the pathogen is intracellular parasite and wherein the intracellular parasite is intracellular parasite belonging to genus Chlamydia.

7. The pharmaceutical composition for vaginitis according to claim 1, wherein a content of the compound represented by the general formula (1) is 1 to 60% by mass with respect to a total amount of the pharmaceutical composition.

8. The pharmaceutical composition for vaginitis according to claim 1, further comprising 40 to 99% by mass of an arbitrary component for preparing a pharmaceutical preparation.

9. The pharmaceutical composition for vaginitis according to claim 1, wherein the pharmaceutical composition is a suppository, a tablet, or a gel.

10. An antiprotozoal agent, comprising a compound represented by a general formula (1) as an active ingredient.

11. The antiprotozoal agent according to claim 10, wherein the protozoa are protozoa belonging to genus Trichomonas.

12. An anti-intracellular parasite agent, comprising a compound represented by a general formula (1) as an active ingredient.

13. The anti-intracellular parasite agent according to claim 12, wherein the intracellular parasite is intracellular parasite belonging to genus Chlamydia.

14. A method for treating vaginitis, comprising administering a pharmaceutical composition comprising a compound represented by the following general formula (1) to a subject in need thereof: 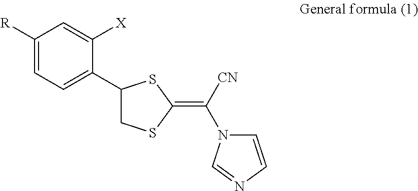
wherein, R represents a halogen atom or a hydrogen atom, and X represents a halogen atom.

15. The method according to claim 14, wherein the compound represented by the general formula (1) is luliconazole or lanoconazole:

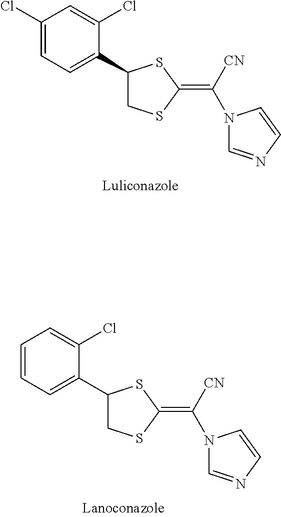

16. The method according to claim 14, wherein the vaginitis is caused by a pathogen selected from the group consisting of intracellular parasite, protozoa, and fungus.

17. The method according to claim 16, wherein the pathogen are protozoa and wherein the protozoa belong to genus Trichomonas.

18. The method according to claim 16, wherein the pathogen are fungus and wherein the fungus is fungus belonging to genus Candida and/or fungus belonging to genus Aspergillus.

19. The method according to claim 16, wherein the pathogen is an intracellular parasite and wherein the intracellular parasite is intracellular parasite belonging to genus Chlamydia.

20. The method according to claim 14, wherein a content of the compound represented by the general formula (1) is 1 to 60% by mass with respect to a total amount of the pharmaceutical composition.
